(19) United States
(12) Patent Application Publication
(10) Pub. No.: US 2018/0185370 A1
(43) Pub. Date: Jul. 5, 2018

(54) PYRAZINOQUINAZOLINONE DERIVATIVES WITH ANTIBACTERIAL ACTIVITY, METHODS AND USES THEREOF

(71) Applicant: UNIVERSIDADE DO PORTO, Porto (PT)

(72) Inventors: Vítor Manuel DE OLIVEIRA E VASCONCELOS, Porto (PT); Lucinda Janete DA SILVA BESSA, Vila Real (PT); Anake KIJJOA, Gondomar (PT); Suradet BUTTACHON, Porto (PT); Tida DETHOUP, Bangkok (TH); Paulo Manuel RODRIGUES MARTINS DA COSTA, S. Pedro do Sul (PT)

(21) Appl. No.: 15/739,353

(22) PCT Filed: Jun. 23, 2016
(86) PCT No.: PCT/IB2016/053750

§ 371 Date: Dec. 22, 2017

(30) Foreign Application Priority Data
Jun. 25, 2015 (PT) 108583

(51) Int. Cl. A61K 31/519; A61K 31/43; C07D 519/00; A61P 31/04; A61K 31/431; A61K 45/06

(52) U.S. Cl. CPC A61K 31/519; A61K 31/43; A61K 45/06; A61P 31/04; A61K 31/431; C07D 519/00

(57) ABSTRACT

The present disclosure relates to compounds and compositions displaying antibacterial activity, particularly against Gram-positive bacteria. The compounds comprise a class of pyrazinoquinazolinone derivatives, including neofiscalin A and fiscalin C, or pharmaceutically acceptable salts, esters, solvates or prodrugs thereof. The present disclosure further relates to methods for treating a patient with a bacterial infection with the disclosed compounds and compositions.

### CROSS REFERENCE TO RELATED APPLICATIONS

This application is a U.S. National Stage Application under 35 U.S.C. § 371 of International Patent Application No. PCT/IB2016/053750, filed Jun. 23, 2016, which claims priority to Portugal Application No. 108583, filed Jun. 25, 2015, both of which are hereby incorporated by reference in their respective entireties.

### TECHNICAL DOMAIN

The present solution relates to medical compounds. The solution discloses pyrazinoquinazolinone derivatives, in particular neofiscalin A and fiscalin C, with antibacterial activity, namely against Gram-positive bacteria.

### TECHNICAL BACKGROUND

Antibiotic resistance is a serious threat to public health worldwide, leading to increased healthcare costs, prolonged hospital stays, treatment failures and deaths (European Centre for Disease Prevention and Control (ECDC). Surveillance Report: Antimicrobial resistance surveillance in Europe 2013. In the last decades, the treatment of infections caused by Gram-positive bacteria has been particularly problematic, when caused by multidrug-resistant organisms, such as methicillin-resistant staphylococci, penicillin- and erythromycin-resistant pneumococci, and vancomycin-resistant enterococci.

Multidrug-resistant Gram-positive bacteria are responsible for a large percentage of infections causing higher morbidity and mortality worldwide. Among these pathogens, methicillin-resistant Staphylococcus aureus (MRSA) and vancomycin-resistant Enterococcus faecalis (VRE) are the ones with great impact in public health, arousing significant concern and health care costs (Woodford, N., Livermore, D. M. 2009. Infections caused by Gram-positive bacteria: a review of the global challenge. J. Infect. 59:S4-16).

Moreover, staphylococci and enterococci have great ability to form biofilms, being recognized as the most frequent causes of biofilm-associated infections, especially biofilms on indwelling medical devices. Bacteria organized within biofilms are more tolerant to antimicrobial agents than their planktonic counterparts. The acquisition and spread of resistance mechanisms and the strategic ability to form a biofilm are putting at risk the usefulness of antibiotics currently available. This situation is complicated by the fact that there have been few classes of antimicrobial agents developed in the last 30 years; and there are no new classes of antimicrobial agents in the pipeline.

Presently, the main strategy to combat the dearth of new effective antibiotics is relying on the chemical modification of known classes of antibiotics.

These facts are disclosed in order to illustrate the technical problem addressed by the present disclosure.

### BRIEF DESCRIPTION

Recent years have seen increasing attention drawn to the continued emergence of multidrug-resistant bacteria. Presently, the main strategy to combat the dearth of new effective antibiotics is relying on chemical modification of known antibiotics. However, if the chemical modification of known antibiotics fails, these new classes of compounds coming from natural sources can be of great interest in the research for new antibiotics as well as in their development.

Nature is an endless source of bioactive compounds, indeed, history proves that natural products were the main source of antibiotics in the past (Butler, M. S., Buss, A. D. 2006. Natural products—the future scaffolds for novel antibiotics? Biochem. Pharmacol. 71:919-929). Thus, the isolation and identification of new naturally occurring compounds from organisms and testing them for antimicrobial susceptibility could pave the way to find potential antimicrobial drugs or at least templates for the development of new antibiotics.

The key strategy in combating drug resistance may be, therefore, the discovery and development of new classes of antibiotics because novel chemical structures that are active against unexploited bacterial targets are less likely to be subjected to existing compound-based or target based resistance mechanisms. The novelty of this disclosure is that none of the antibiotics currently in use belongs to this chemical class of compounds. Furthermore no single antibiotic available in the market was developed from marine natural products until now.

This disclosure is directed to pyrazinoquinazolinone derivatives, in particular neofiscalin A and fiscalin C. The neofiscalin A was, in particular, isolated from the marine fungus Neosartorya siamensis (KUFA 0017) and the terrestrial fungus N. siamensis (KUFC 6349); and fiscalin C was isolated, in particular, from the soil fungus N. siamensis (KUFC 6349).

Neofiscalin A consistently showed a MIC value of 8 μg/ml against S. aureus and E. faecalis strains, including multidrug-resistant isolates. Fiscalin C consistently showed an unexpected synergistic effect with oxacillin against methicillin-resistant Staphylococcus aureus (MRSA).

The advantages associated with this disclosure are:
the pyrazinoquinazolinone derivatives are produced by culturable marine or/and terrestrial fungi, and therefore may be obtained in large scale for further in vivo studies as well as for further modifications in order to ameliorate their antibacterial activity;
the ability of neofiscalin A to inhibit not only planktonic bacteria but also to hamper the biofilm formation, suggests that it can be an effective option to overcome acquired as well as phenotypical resistances;
the synergistic effect between fiscalin C and oxacillin against methicillin-resistant Staphylococcus aureus (MRSA) bacteria;
the pyrazinoquinazolinone derivatives, in particular neofiscalin A and fiscalin C, are not structurally and/or chemically related with the classes of any of the antibiotics currently available in the market, consequently they may be more protected from resistance acquired by genetic competition and may be exploited as antibacterial agents in human and veterinary medicines.

A pyrazinoquinazoline derivative is any compound comprising a pyrazine ring fused to a quinazoline ring.

The minimum inhibitory concentration (MIC) is the lowest concentration of a compound that inhibits the visible growth of a given strain.

The minimum bactericidal concentration (MBC) is the lowest concentration of a compound required to kill a particular bacterial strain.

The biofilm inhibitory concentration (BIC) is the lowest concentration of the compound that inhibits the growth of the biofilm, which is confirmed by no increase in the optical density of the supernatant fluid compared with the initial reading.

The biofilm eradication concentration (BEC) is the lowest concentration that prevents regrowth of bacteria from the pre-treated biofilm.

The present disclosure describes pyrazinoquinazolinone derivatives isolated from fungi, with antibacterial activity against Gram-positive bacteria. In particular, the present disclosure describes compounds of the formula I:

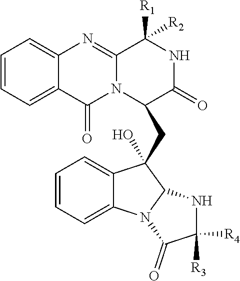

wherein R₁, R₂, R₃ and R₄ are independently selected from the following list: H, C₁-C₁₀ alkyl, C₂-C₁₀ alkenyl, C₁-C₁₀ aryl; providing that R₁ is equal to R₃ or R₃ is equal to R₄; and R₂ is selected from the following list: C₁-C₁₀ alkyl; or pharmaceutically acceptable salt, ester, solvate or prodrug thereof, for use in medicine.

In the present disclosure, the term “alkyl” relates to a linear, cyclic or branched hydrocarbon group comprising from 1 to 10 carbon atoms, preferably from 1 to 6 carbon atoms, in particular from 1 to 3 carbon atoms. Among alkyl groups can be cited for instance the methyl, ethyl, n-propyl, isopropyl, n-butyl, sec-butyl, tert-butyl, n-pentyl, n-hexyl and cyclohexyl groups.

The term “alkenyl” relates to an alkyl group as defined above, further comprising at least one C═C double bond.

The term “aryl” relates to a group comprising at least one planar ring comprising a conjugated π system made of double bonds and/or non-bonding doublets, wherein each atom of the ring comprises a p orbital, the p orbitals overlay each other, and the derealization of the π electrons lowers the molecule energy. Preferably, the aryl group is a hydrocarbon aryl group. Preferably, an aryl group is selected from the group consisting of phenyl, pyridinyl, pyrimidinyl, pyrazinyl, pyridazinyl, triazinyl, furanyl, thiophenyl, pyrrolyl, imidazolyl, thiazolyl, oxazolyl, isoxazolyl, dihydroisoxasolyl, triazolyl, diazinyl, tetrazinyl, pyrazolyl and naphthyl groups.

In an embodiment, the C₁-C₁₀ alkyl is preferably a C₁-C₆ alkyl, more preferably a C₁-C₃ alkyl.

In an embodiment, the alkyl is a non-linear alkyl.

In an embodiment, R₁ and R₃ of the compound of formula I are H.

In an embodiment, R₃ and R₄ of the compound of formula I are methyl.

In an embodiment, R₁ and R₃ are H, R₂ is CH(CH₃)₂ and R₄ is methyl.

In an embodiment, R₁ is H, R₂ is CH(CH₃)₂, R₄ and R₃ are methyl.

The present disclosure also relates to the use of any of the previously described compounds in the treatment of bacterial infections, wherein the microorganism involved is a Gram-positive bacteria selected from the following list: Staphylococcus spp., Streptococcus spp., Enterococcus spp., Listeria spp., Clostridium spp., Corynebaterium spp., Nocardia spp., Bacillus spp. and mixtures thereof.

The present disclosure also describes a composition comprising any of the compounds previously described in a therapeutically effect amount and a pharmaceutically acceptable excipient.

In an embodiment, the composition further comprises an antibiotic, wherein the antibiotic is a beta-lactam antibiotic, in particular oxacillin, nafcillin, cloxacillin, dicloxacillin, or flucloxacillin, or mixtures thereof.

In an embodiment, the composition previously described is in an oral, injectable, or parental form.

The compositions can be combined with other excipients or active substances used in the context of veterinarian or human medicine.

The compound or compositions of the present disclosure can be administered by various routes, including topical, enteral and parenteral. Parenteral administration routes include intra-arterial, intra-articular, intracavitary, intradermal, intralympathic, intramuscular, intrasynovial, intravenous, or subcutaneous. Enteral routes include oral and rectal. Topical routes include application into the skin and mucous membranes.

In a preferred embodiment, the composition is delivered to a patient by oral administration, which can be repeated according to a clinical prescription regime.

Dosage of the composition can be adapted to the administration route, as well as to the patient profile, including age, gender, condition, disease progression, or any other phenotypic or environmental parameters.

The composition may be in a solid form such as an amorphous, crystalline or semi-crystalline powder, granules, flakes, pills, capsules and suppositories. Such a solid form can be converted into a liquid form by mixing the solid with a physiologically appropriate liquid such as solvents, solutions, suspensions and emulsions.

In another aspect, the present invention provides a method of treating a patient with a bacterial infection, the method comprising administering an effective amount of compound or composition of the present disclosure to the patient.

In a further aspect, the present invention provides the compound or composition of the present disclosure for use in the treatment of bacterial infections.

Further, the present invention provides the use of compound or composition of the present disclosure in the manufacture of a medicament for the treatment of bacterial infections.

Throughout the description and claims the word “comprise” and variations of the word, are not intended to exclude other technical features, additives, components, or steps. Additional objectives, advantages and features of the solution will become apparent to those skilled in the art upon examination of the description or may be learned by practice of the solution.

### BRIEF DESCRIPTION OF THE DRAWINGS

The following figures provide preferred embodiments for the present disclosure and should not be seen as limiting the scope of the disclosure.

FIGS. 1A-1I: Secondary metabolites isolated from the marine-derived Neosartorya siamensis KUFA 0017 and the soil fungus N. siamensis KUFC 6349 wherein FIG. 1A is 2,4-dihydroxy-3-methylacetophenone; FIG. 1B is chevalone C; FIG. 1C is chevalone B; FIG. 1D is sartorymensin; FIG. 1E is tryptoquivaline; FIG. 1F is nortryptoquivaline; FIG. 1G is 3′-(4oxoquinazolin-3yl) spiro [1H-indole-3,5′-oxolane]-2,2′-dione; FIG. 1H displays tryptoquivaline L (equation i), tryptoquivaline H (equation ii), tryptoquivaline F (equation iii), and tryptoquivaline 0 (equation iv); and FIG. 1I displays fiscalin C (equation v), epi-fiscalin C (equation vi); fiscalin A (equation vii); epi-fiscalin A (equation viii); neofiscalin A (equation ix), and epi-neofiscalin A (equation x).

FIGS. 2A-2B: Chemical structures of neofiscalin A (FIG. 2A) and fiscalin C (FIG. 2B).

FIG. 3: Biomass quantification of biofilms formed by Gram-positive bacteria in the presence of different concentrations (ranging from 2×MIC to ¼×MIC) of neofiscalin A, through the crystal violet assay. Asterisks indicates that the difference between the concrete condition and the control is statistically significant (P<0.05).

FIGS. 4A-4C: Microscopic visualization of 24-h biofilms formed by MRSA and VRE, using the live/dead viability staining. Biofilm formation in absence (FIG. 4A), in the presence of the MIC (FIG. 4B) and ¼× of the MIC (FIG. 4C) of neofiscalin A.

FIG. 5: Metabolic activity of biofilms after treatment with neofiscalin A (16). Quantification of metabolic activity, using MTT, of 24-h preformed biofilms of S. aureus B1 (MRSA) and E. faecalis (VRE) after treatment with several concentrations (up to 25×MIC-200 μg/ml) of neofiscalin A.

FIG. 6: Cytotoxicity of neofiscalin A against a hCMEC/D3 cell line expressed as a percentage of cell viability considering 100% viability in the negative control (1% DMSO).

FIG. 7: Dose-response curve of the haemolytic activity of neofiscalin A against h-RBCs.

### DETAILED DESCRIPTION

In the pursuit of new antibiotics from fungi, both terrestrial and marine-derived fungi, the chemical constituents of a Thai collection of the marine sponge-associated fungus N. siamensis KUFA 0017 were investigated. N. siamensis was isolated from the sea fan Rumphella spp., which was collected from the coral reef of the Similan islands, Phang Nga province, Southern Thailand, by scuba diving at 10 m depth, in April 2010. Briefly, the sea fan tissue was cut into a piece of 0.5 cm×0.5 cm, placed on the malt extract agar (MEA) with 70% sea water and incubated for 28° C. for 7 days. The fungus was identified by morphological features, including the characteristic of ascospores, conidiogenesis and colonies, as well as by sequence analysis of the β-tubulin gene described in the previous report. The pure cultures were deposited as KUFA 0017 at Kasetsart University Fungal Collection, Department of Plant Pathology, Faculty of Agriculture, Kasetsart University, Bangkok, Thailand.

### Extraction of Secondary Metabolites

N. siamensis KUFA 0017 was cultured, in particular for one week in five 90 mm Petri dishes with 25 ml of potato dextrose agar with 70% sea water per dish. In particular, thirty-five 1000 ml Erlenmeyer flasks each containing 200 g of rice and 100 ml of water were autoclaved at 121° C. for 15 min, inoculated with ten mycelium plugs of the fungus and incubated at 28° C. for 30 days. Ethyl acetate, in particular 500 ml, was added to each flask containing the moldy rice and the content was left to macerate for 7 days, and then filtered by filter paper. The two layers were separated using a separatory funnel, and the ethyl acetate solution was evaporated, in particular under reduced pressure to yield 95 g of crude ethyl acetate extract which was dissolved in CHCl₃, in particular 700 ml, and then washed with H₂O, in particular 3×500 ml. The organic layer was, in particular, dried with anhydrous Na₂SO₄, filtered and evaporated under reduced pressure to give, 70 g the crude chloroform extract.

The crude chloroform extract, in particular 30 g, was applied on a column of silica gel, in particular 350 g, and eluted with mixtures of CHCl₃-petrol and CHCl₃-Me₂CO, 250 ml fractions were collected, in particular, as follows: fractions 1-10 (CHCl₃-petrol, 1:1), fractions 11-69 (CHCl₃-petrol, 7:3), fractions 70-204 (CHCl₃-petrol, 9:1), fractions 205-260 (CHCl₃-Me₂CO, 9:1), fractions 261-400 (CHCl₃-Me₂CO, 4:1), fractions 401-455 (CHCl₃-Me₂CO, 7:3).

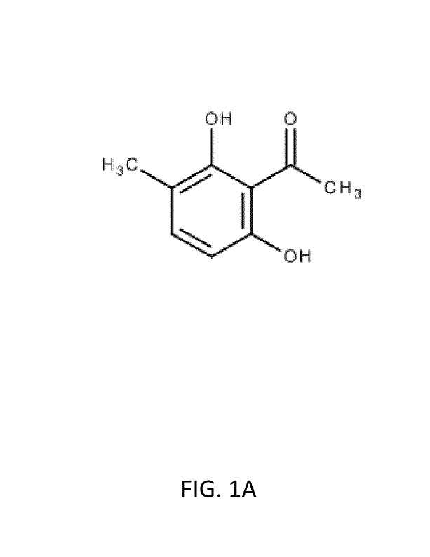

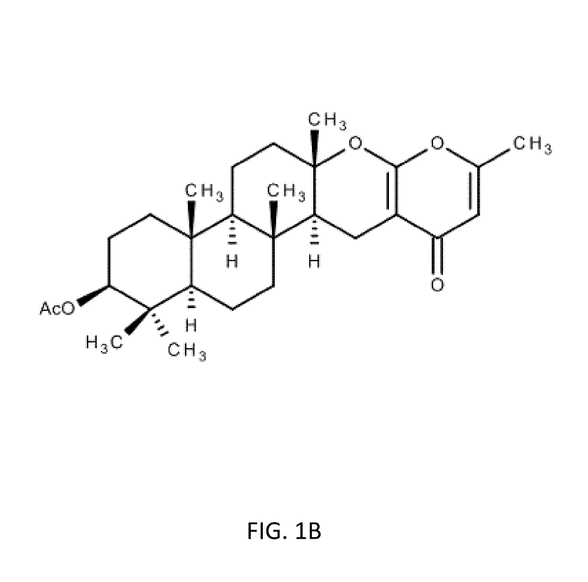

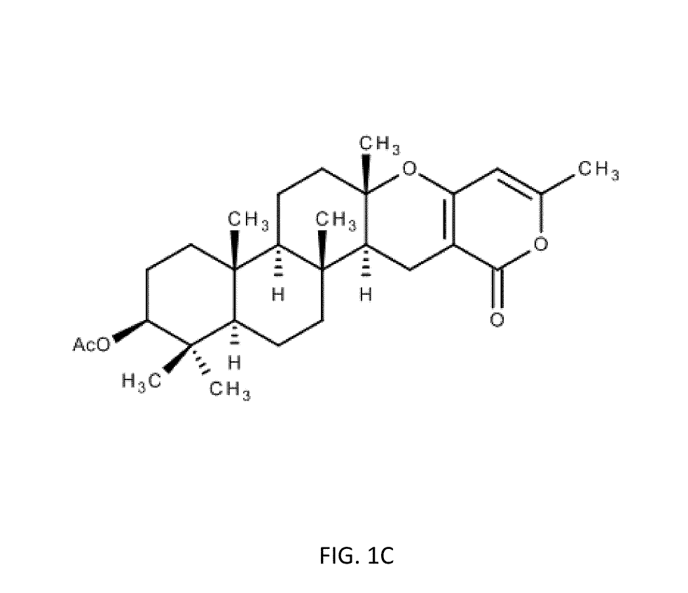

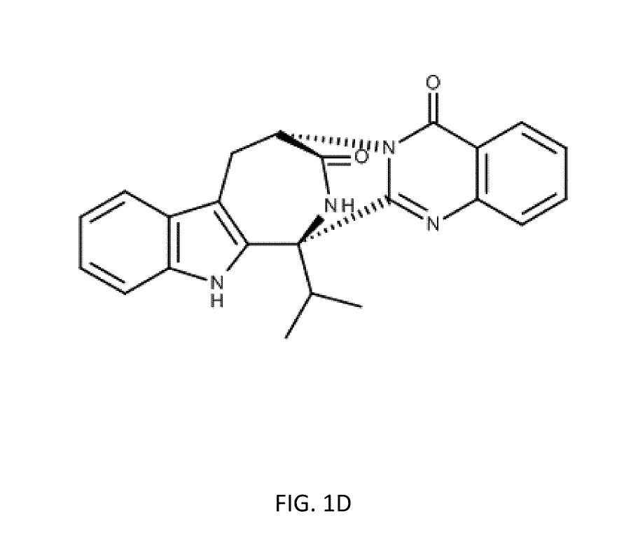

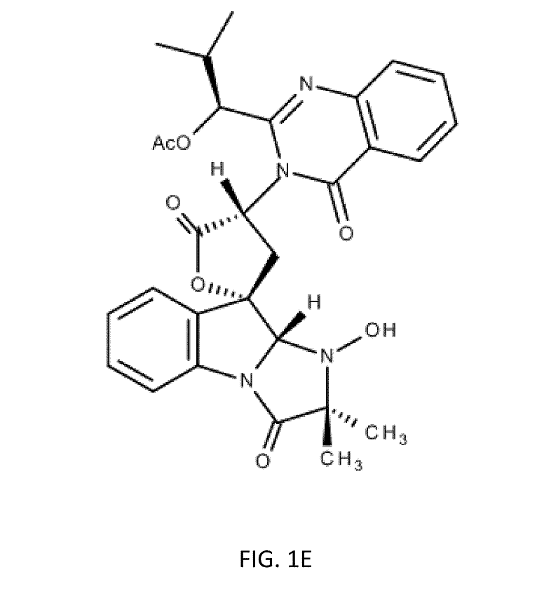

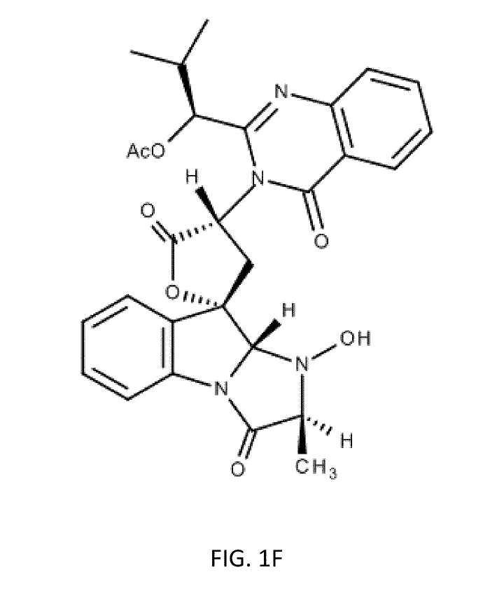

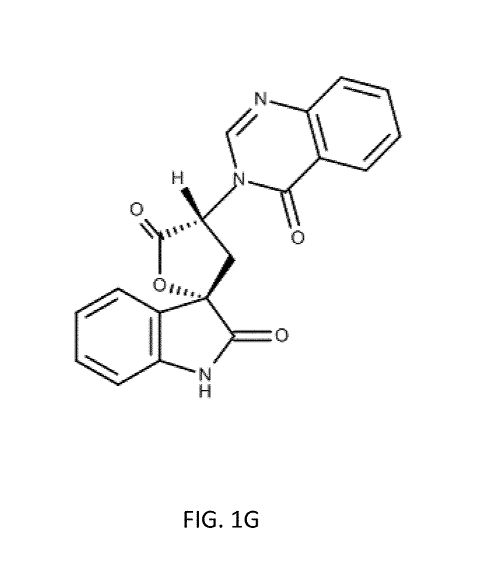

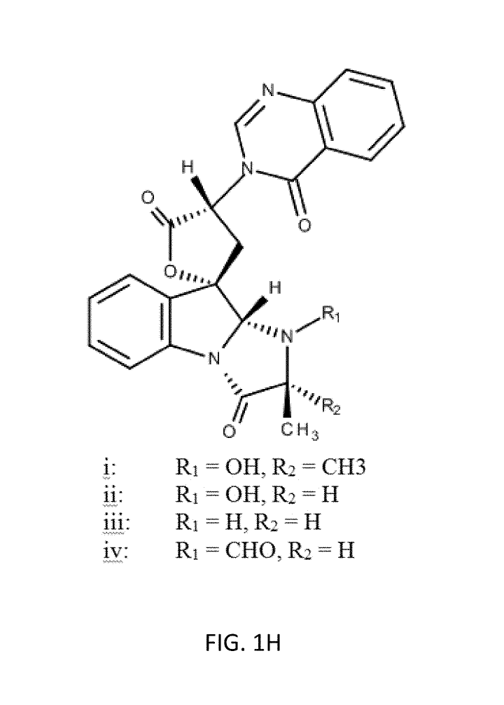

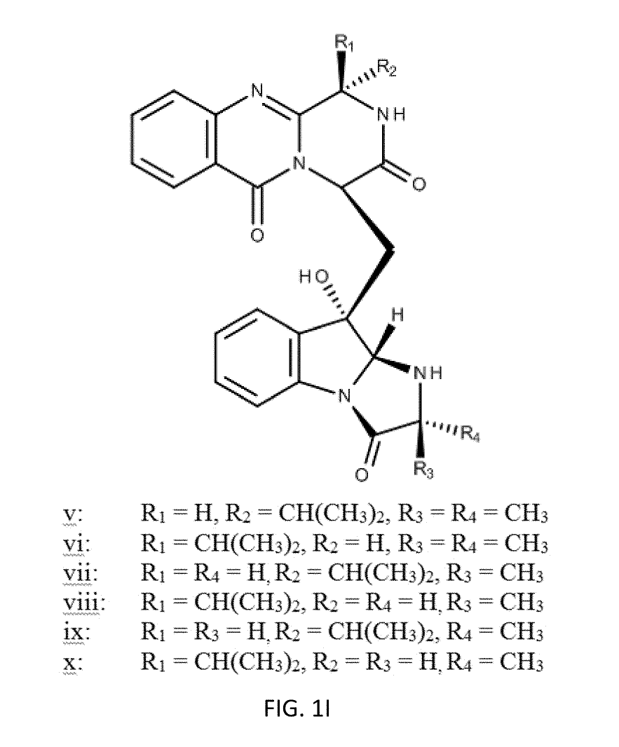

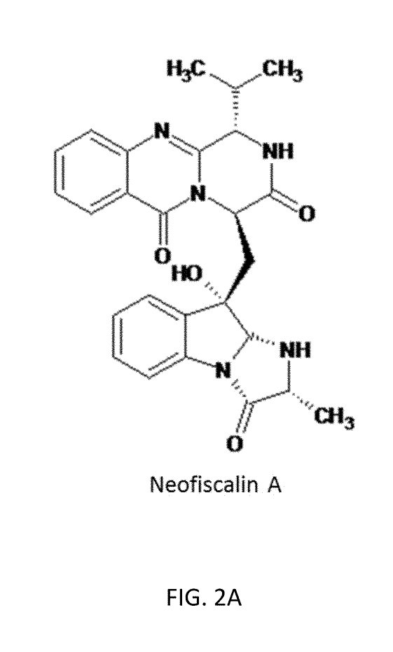

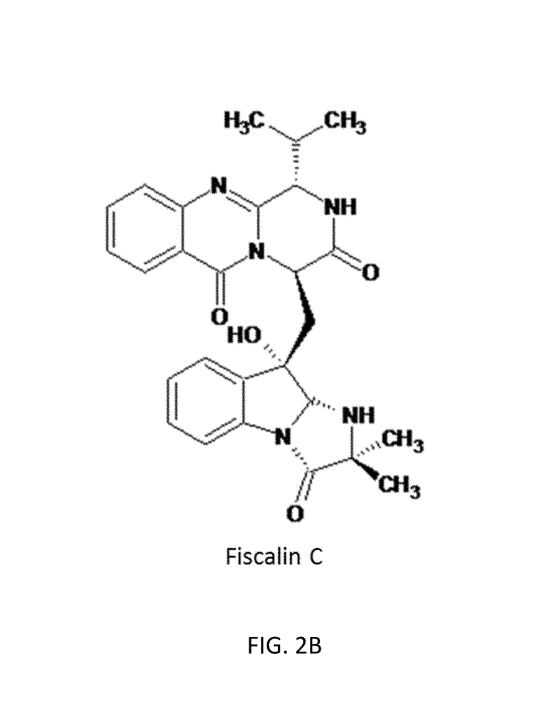

Fractions 149-152 were combined, in particular 46 mg, and purified by TLC, in particular Si Gel, CHCl₃-EtOAc-petrol-HCO₂H, 9:0.5:0.5:0.1 to give 15 mg of 2,4-dihydroxy-3-methylacetophenone (see FIG. 1A).

Fractions 153-191 were, in particular, combined and crystallized in a mixture of petrol and CHCl₃ to give 192 mg of 2,4-dihydroxy-3-methylacetophenone.

Fractions 212-215 were, in particular, combined (1.52 g), applied on a Si Gel column (20 g) and eluted with mixtures of petrol-CHCl₃ and CHCl₃-Me₂CO, wherein 100 ml sub-fractions (sfrs) were collected as follows: sfrs 1-15 (CHCl₃-petrol, 7:3), 16-45 (CHCl₃-petrol, 9:1), 46-50 (CHCl₃-Me₂CO, 9:1), 51-76 (CHCl₃-Me₂CO, 7:3). Sfr 8-55 were combined and recrystallized in a mixture of petrol and CHCl₃ to give additional 223 mg of 2,4-dihydroxy-3-methylacetophenone. Sfrs 56-70 were combined (360 mg) and purified by TLC (Si Gel, CHCl₃-Me₂CO—HCO₂H, 8:2:0.1) to give 26.7 mg of tryptoquivaline, 32.2 mg of nortryptoquivaline and 106 mg of chevalone C (see FIG. 1B).

Fraction 217, in particular 360 mg of fraction 217, was crystallized in a mixture of petrol and CHCl₃ to give 153 mg of chevalone C. The mother liquor, in particular 206 mg, was combined with fraction 216 and fractions 218-223 (1.27 g), and purified by TLC (Si Gel, CHCl₃-Me₂CO—HCO₂H, 8:2:0.1) to give 24.2 mg of nortryptoquivaline, 569 mg of chevalone C, and 8.8 mg of tryptoquivaline L.

Fractions 264-272 were combined, in particular 1.22 g, and crystallized in Me₂CO to give 52.8 mg of tryptoquivaline L.

Fractions 273-282 were combined, in particular 1.02 g, and crystallized in Me₂CO to give white solid (218 mg) which was purified by TLC (Si Gel, CHCl₃-Me₂CO—HCO₂H, 7:3:0.1) to give 2.5 mg of tryptoquivaline L and 11.6 mg of tryptoquivaline H.

Fractions 283-309 were combined, in particular 1.64 g, and crystallized in a mixture of CHCl₃ and MeOH to give 263 mg of tryptoquivaline H. The mother liquor of fractions 264-272 and of fractions 283-309 were combined with fractions 224-263 (4.55 g), applied on a Si Gel column (30 g) and eluted with mixtures of CHCl₃ and Me₂CO, wherein 100 ml sub-fractions were collected as follows: sfrs 1-9 (CHCl₃), 10-19 (CHCl₃-Me₂CO, 9:1), 20-35 (CHCl₃-Me₂CO, 7:3). Sfrs 11-14 were combined (883 mg) and crystallized in MeOH to give 69.7 mg of fiscalin A. Sfrs 19-30 were combined and crystallized in MeOH to give 34.4 mg of epi-fiscalin C. Sfrs 31-35 were combined and crystallized in MeOH to give 246 mg of tryptoquivaline F.

Fractions 310-345 were combined (990 mg) and crystallized in Me₂CO to give 602 mg of white precipitate which was further purified by TLC (Si Gel, CHCl₃-Me₂CO—HCO₂H, 7:3:0.1) to give 10.3 mg of neofiscalin A, 12.6 mg of fiscalin A, 5.6 mg of epi-fiscalin C, 7.8 mg of epi-neofiscalin A and 8.4 mg of epi-fiscalin A. The mother liquor (539 mg) was applied on a Si Gel column (30 g) and eluted with mixtures of CHCl₃ and Me₂CO, wherein 100 ml sub-fractions were collected as follows: sfrs 1-19 (CHCl₃), 20-23 (CHCl₃-Me₂CO, 9:1), 25-28 (CHCl₃-Me₂CO, 7:3), 29 (MeOH). Sfrs 7-9 were combined (42.1 mg) and purified by TLC (Si Gel, CHCl₃-Me₂CO—HCO₂H, 8:2:0.1) to give 12.1 mg of epi-neofiscalin A and 5.4 mg of epi-fiscalin A. Sfrs 10-16 were combined (27.6 mg) and purified by TLC (Si Gel, CHCl₃-Me₂CO—HCO₂H, 8:2:0.1) to give 7.6 mg of epi-neofiscalin A and 3.8 mg of tryptoquivaline F. Sfrs 17-19 were combined (65.6 mg) and purified by TLC (Si Gel, CHCl₃-Me₂CO—HCO₂H, 7:3:0.1) to give 12.8 mg of 3′-(4-oxoquinazolin-3-yl) spiro[1H-indole-3, 5′-oxolane]-2, 2′-dione, 2.6 of mg epi-neofiscalin A and 1.4 mg of epi-fiscalin A. Sfrs 20-23 were combined (93 mg) and recrystallized in MeOH to give 27.9 mg of epi-neofiscalin A. Sfr 24 (143.5 mg) was crystallized in MeOH to give 40.5 mg of white solid which was purified by TLC (Si Gel, CHCl₃-Me₂CO—HCO₂H, 7:3:0.1) to give 10.4 mg of epi-neofiscalin A, 11.5 mg of epi-fiscalin A and 8.6 mg of tryptoquivaline F. Sfrs 25-28 were combined (51.9 mg) and crystallized in MeOH to 7.6 mg of epi-fiscalin A.

Fractions 346-356 were combined (286.1 mg) and crystallized in Me₂CO to give white precipitate (75 mg) which was further purified by TLC (Si Gel, CHCl₃-Me₂CO—HCO₂H, 7:3:0.1) to give 4.8 mg of neofiscalin A, 28.8 mg of fiscalin A, 1.4 mg of epi-fiscalin C, 36.6 mg of epi-neofiscalin A, and 11.8 mg of epi-fiscalin A.

Fractions 357-368 were combined (39.3 mg) and crystallized in Me₂CO to give white precipitate (162.7 mg) which was further purified by TLC (Si Gel, CHCl₃-Me₂CO—HCO₂H, 7:3:0.1) to give 0.6 mg fiscalin A, 13.0 mg of epi-neofiscalin A, and 73.3 mg of epi-fiscalin A.

Fractions 369-403 were combined (1.7 g) and crystallized in MeOH to give 360.7 mg of epi-fiscalin A.

Fractions 404-419 were combined (252.3 mg) and crystallized in MeOH to give 126.7 mg of tryptoquivaline F. The mother liquor of fractions 346-419 were combined (1.47 g), applied on a Si Gel column (30 g) and eluted with mixtures of CHCl₃-Me₂CO, and MeOH, wherein 100 ml sub-fractions were collected as follows: sfrs 1-22 (CHCl₃-Me₂CO, 9:1), 23-29 (CHCl₃-Me₂CO, 7:3), 40-45 (Me₂CO), 46 (MeOH). Sfrs 32-39 (374.4 mg) were combined and crystallized in MeOH to give 32.8 mg of epi-fiscalin A. Sfrs 40-45 (155.5 mg) were combined and purified by TLC (Si Gel, CHCl₃-Me₂CO—HCO₂H, 3:2:0.1) to give 6.8 mg of tryptoquivaline F.

Therefore, the ethyl acetate extract of the marine sponge-associated fungus N. siamensis KUFA 0017 furnished, besides 2,4-dihydroxy-3-methylacetophenone and chevalone C, the indole alkaloids 3′-(4-oxoquinazolin-3-yl) spiro[1H-indole-3,5′-oxolane]-2, 2′-dione, nortryptoquivaline, tryptoquivaline, tryptoquivalines L, H and F, fiscalin A, epi-fiscalin A, neofiscalin A, epi-neofiscalin A and epi-fiscalin C. The structures of the isolated compounds were established based on the HRMS, 1D and 2D NMR analysis, as well as by comparison of their NMR data to those previously described by (Buttachon, S., Chandrapatya, A., Manoch, L., Silva, A., Gales, L., Bruyère, C., Kiss, R., Kijjoa, A. 2012. Sartorymensin, a new indole alkaloid, and new analogues of tryptoquivaline and fiscalins produced by Neosartorya siamensis (KUFC 6349). Tetrahedron 68:3253-3262).

Compounds in FIGS. 1E, 1G, 1H (equations i-iii), and 11 (equations vi-x), together with tryptoquivaline O (see FIG. 1H, equation iv), fiscalin C (see FIG. 1I, equation v) and sartorymensin (see FIG. 1D) previously isolated from the soil fungus N. siamensis KUFC 6349 (Buttachon, S., Chandrapatya, A., Manoch, L., Silva, A., Gales, L., Bruyère, C., Kiss, R., Kijjoa, A. 2012. Sartorymensin, a new indole alkaloid, and new analogues of tryptoquivaline and fiscalins produced by Neosartorya siamensis (KUFC 6349) Tetrahedron 68:3253-3262), were evaluated for their antibacterial activity against Gram-positive and Gram-negative bacteria as well as for synergy with antibiotics against multidrug-resistant bacteria.

TABLE 1
Summary of the isolated compounds per fungus.

| | Marine-derived | Soil fungus |
|---|---|---|
| | N. siamensis KUFA 0017 | N. siamensis KUFC 6349 |
| Com- | Compounds of FIGS. 1A, 1B, | Compounds of FIGS. 1A, 1D, |
| pounds | 1E-1G, 1H (equations i-iii), | 1E, 1G, 1H (equations i-iv), |
| | and 1I (equations vi-x) | and 1I (equations v-x) |

General experimental procedures. The melting points were determined on a Bock monoscope and are uncorrected. Optical rotations were determined on an ADP410 Polarimeter. Infrared spectra were recorded on an ATT Mattson Genesis Series FTIR™ using WinFIRST Software. ¹H and ¹³C NMR spectra were recorded at ambient temperature on a Bruker AMC instrument operating at 500.13 and 125.8 MHz, respectively. High resolution mass spectra were measured with a Waters Xevo QToF mass spectrometer coupled to a Waters Aquity UPLC system. A Merck silica gel GF₂₅₄ was used for preparative TLC, and a Merck Si gel 60 (0.2-0.5 mm) was used for analytical chromatography.

Chemical synthesis of the above-mentioned compounds, in particular neofiscalin A. Neofiscalin A can be synthetized by a similar process to the one described for fumiquinazolines A, B, C, E, H, and I (Snider, B. B., Zeng, H. 2003. Total Synthesis of (−)-Fumiquinazolines A, B, C, E, H, and I. Approaches to the synthesis of fiscalin A. J. Org. Chem. 68:545-563). Neofiscalin A has two polycyclic moieties linked by a methylene unit; therefore, the two portions can be prepared separately. The synthesis of the quinazolinone rings system has been already described using the Ganesan-Mazurkiewicz cyclization. The imidazoindolone rings system of neofiscalin A may be prepared from the amino acid glycine, that will allow to retain the stereochemistry essential for the activity, and from the 3-methylindole. Neofiscalin A has a hydroxyl group that allows molecular modifications by nucleofilic substitution. Nonetheless, it is not known if the analogs of neofiscalin A with other substituents such as alkyl, alkenyl or aryl groups, besides the hydroxyl substituent on C-19, the isopropyl on C-3 and the methyl on C-22, can also be active or not. Taking into account the complexity of the molecule, and based on other natural products with antimicrobial activities, molecular simplification/modification can result in derivatives which are not only more potent but also more selective and specific than the naturally occurring compound, as happens in the case of macrolides, aminoglycosides and aminopenicillins.

Bacterial strains and growth conditions. Five reference strains, Staphylococcus aureus ATCC 25923, Bacillus subtilis ATCC 6633, E. faecalis ATCC 29212, Escherichia coli ATCC 25922 and Pseudomonas aeruginosa ATCC 27853, and three multidrug-resistant isolates from environment, S. aureus B1 (a MRSA isolated from public buses), Enterococcus faecalis W1 (a VRE isolated from river water) and E. coli G1 (isolated from seagull feces) were used. Bacteria were grown overnight at 37° C. in Mueller-Hinton agar (MH—BioKar diagnostics, Allonne, France) prior to obtain be used in the subsequently described bioassays.

Antibacterial susceptibility testing. The minimum inhibitory concentration (MIC) values of the compounds of FIGS. 1D, 1E, 1G, 1H (equation i-iv) and 11 (equations v-x) were determined, in particular, by a broth microdilution technique, following the recommendations of the Clinical and Laboratory Standards Institute. Stock solutions of 10 mg/ml in DMSO were used to prepare in-test concentrations ranging from 2 to 256 μg/ml. Ciprofloxacin in the concentration range from 0.03125 to 16 μg/ml was used, in particular, as control drug in the experiment. The MIC was defined as the lowest concentration of compound that has inhibited the visible growth. The minimum bactericidal concentration (MBC) was determined by spreading on MH plates, 10 μL collected from the wells corresponding to the MIC and to higher concentrations, with further incubation for 24 h at 37° C.; the lowest concentration at which no growth occurred on MH was defined as the MBC.

Synergistic studies between compounds and antibiotics. A screening susceptibility test to assess the combined effect between the compounds and antibiotics was conducted using the disc diffusion method on MH, against three multidrug-resistant isolates, E. coli G1, S. aureus B1 and E. faecalis W1. A set of antibiotic discs (Oxoid, Basingstoke, England) was selected based on the resistance of the isolates towards those antibiotics. Based on the results of the previous assay, an unexpected synergetic effect between fiscalin C (see FIG. 1I, equation v) and oxacillin (Sigma-Aldrich, St. Louis, Mo., USA) was determined using, in particular, a broth microdilution Checkerboard method against the MRSA isolate (S. aureus B1). The fractional inhibitory concentration (FIC) was calculated as follows: FIC of drug A (FIC A)=MIC of drug A in combination/MIC of drug A alone, and FIC of drug B (FIC B)=MIC of drug B in combination/MIC of drug B alone. The FIC index (ΣFIC), calculated as the sum of each FIC, was interpreted as follows: ΣFIC 0.5, synergy; 0.5<ΣFIC 4, no interaction; 4<ΣFIC, antagonism.

Evaluation of the ability of neofiscalin A (see FIG. 1I, equation ix) to inhibit the biofilm formation. The efficacy of neofiscalin A was also evaluated in hampering the biofilm formation. Neofiscalin A, in particular, at concentrations of 2×MIC, MIC, ½×MIC and ¼×MIC was added to bacterial suspensions of 1×10⁶ CFU/ml in Tryptic Soy Broth (TSB—BioKar diagnostics, Allonne, France) of S. aureus ATCC 25923, E. faecalis ATCC 29212, S. aureus B1 and E. faecalis W1. Bacterial suspensions in absence of the compound were used as controls. Biofilm formation was induced, in particular, in 96-well flat-bottomed microtiter plates (200 μL/well) for 24 h at 37° C. After that time, biofilm biomass was quantified through the crystal violet assay as follows: the planktonic phase was discharged, then the biofilm was stained with 0.5% crystal violet for 5 min, rinsed with water, air dried and eluted with acetic acid 33% (v/v). The optical density was measured at 595 nm (0D595) using a microplate reader (iMark™ microplate absorbance reader, Bio-Rad Laboratories, Hercules, Calif., USA). Two independent experiments were performed in triplicate for each experimental condition. The statistical significance of the difference between biofilm controls and biofilms in the presence of different concentrations of compounds was evaluated using Student's t test. In both cases, probability levels <0.05 were considered statistically significant. Additionally, a microscopic visualization of the biofilms of S. aureus B1 and E. faecalis W1 was performed, in particular, using the Live/Dead BacLight viability kit (Life Technologies—Molecular Probes, Carlsbad, Calif., USA). Biofilms were formed, in particular, in 35-mm diameter polystyrene plates using TSB (control) and TSB supplemented with the MIC or ¼×MIC of neofiscalin A. After 24 h at 37° C., the planktonic phase was removed from each plate and the biofilms were washed with PBS, stained with the appropriate mixture of SYTO 9 and propidium iodide stains and incubated for 20 min at room temperature, in particular 20-25° C., in the dark; then, biofilms were rinsed and examined under a fluorescence microscope (BX41 Microscope, Olympus America Inc., Center Valley, Pa., USA). Images were recorded at an emission wavelength of 500 nm for SYTO 9 (green fluorescence) and of 635 nm for propidium iodide (red fluorescence).

Determination of neofiscalin A biofilm inhibitory and biofilm eradication concentrations. The efficacy of neofiscalin A on 24-h mature biofilms of S. aureus B1 and E. faecalis W1 was also evaluated, in particular, by determining the BIC and the BEC. After the formation of biofilms for 24 h, as above described, the planktonic cells were gently removed and the wells were rinsed once and then filled with different concentrations of neofiscalin A ranging from the MIC value up to 12×MIC. The optical density at 595 nm was measured at time 0 and after incubation for 24 h at 37° C. The BIC was determined as the lowest concentration of the compound inhibiting growth in the supernatant fluid, confirmed by no increase in the optical density compared with the initial reading. The BEC was determined as the lowest concentration that prevented regrowth of biofilm cells on tryptic soy agar plates.

Evaluation of the biofilm metabolic activity after treatment with different concentrations of neofiscalin A. Twenty four-hours biofilms of S. aureus B1 and E. faecalis W1 formed as above described were subsequently treated with high concentrations of neofiscalin A, in particular up to 25×MIC. After 24 h of incubation at 37° C., the bacterial metabolic activity of biofilms was quantified using the 3-(4,5-dimethylthiazole-2-yl)-2,5-diphenyl tetrazolium bromide (MTT, Sigma-Aldrich, St Louis, USA). MTT is used to assess cell viability as a function of redox potential. Actively respiring cells convert the water-soluble MU to an insoluble purple formazan. The formazan is then solubilized and its concentration determined by optical density. In this assay, biofilms were treated with MU (0.5 mg/ml) for 2 h at 37° C. in the dark. DMSO was used to extract the formazan dye product and then the absorbance at 550 nm was measured.

Cytotoxicity and hemolytic assays for neofiscalin A. The cytotoxicity of neofiscalin A was evaluated, in particular, using the normal human brain microvascular endothelial cell line hCMEC/D3. Cells were cultivated in DMEM Glutamax supplemented with 10% fetal bovine serum (FBS), 2.5 μg/ml fungizone and penicillin-streptomycin (100 IU/ml and 100 μg/ml, respectively) and incubated at 37° C. in a humidified atmosphere with 5% of CO₂. Cellular viability was evaluated, in particular, by the reduction of the MU. Cells were seeded in 96-well culture plates in 100 μl of medium at 3.3×10⁴ cells/ml. After 24 h of adhesion cells were incubated in new medium with neofiscalin A, in particular, at concentrations of 20, 10 and 5 μg/ml; 1% DMSO as the negative control and 1% Triton X-100 as the positive control. After 24 and 48 h of incubation, cells were exposed, in particular, to 10 μl of 0.5 mg/ml MU for 3 h, at 37° C. After incubation, the purple formazan salts were dissolved in 100 μl DMSO, and the absorbance measured at 550 nm. These tests were run in triplicate and averaged. Cytotoxicity was expressed as the percentage of cell viability considering 100% viability in the negative control.

The hemolytic activity of neofiscalin A was assessed in human red blood cells (h-RBCs). Briefly, fresh human blood, collected with EDTA, was centrifuged at 3000 rpm for 5 min and washed three times with PBS pH 7.4. Then, red blood cells were diluted to 5% in PBS and incubated with different dilutions of the compound ranging from 2 to 200 μg/ml. After 1 h of incubation at 37° C., the suspensions were sedimented by centrifugation and the release of hemoglobin was measured by reading the absorbance (A) at 405 nm and compared with a 0% hemolysis control (PBS) and a 100% hemolysis control (PBS with 1% v/v Triton X-100). The percentage of hemolysis was calculated using the following equation: Hemolysis (%)=[(Asample−A0% lysis control)/(A100% lysis control−A0% lysis control)]×100.

Compounds of FIGS. 1D, 1E, 1G, 1H (equations i-iv), and 1I (equations v-x) were tested for their antibacterial activity against bacterial reference strains and environmental multidrug-resistant isolates, and their MIC and MBC values (when determined) are shown in Tables 2 and 3, respectively. Interestingly, only neofiscalin A presented antibacterial activity against Gram-positive bacteria while the rest of the indole alkaloids was inactive at the highest concentration tested (256 μg/ml) (Table 2). Neofiscalin A consistently showed a low value of MIC (8 μg/ml) against S. aureus and E. faecalis strains. However the MBC of Neofiscalin A was usually four times higher than the MIC (Table 3).

TABLE 2
Minimum inhibitory concentration-MIC-values (μg/ml) against several bacterial
reference and multidrug-resistant strains.

| | | | P. | | | | |
|---|---|---|---|---|---|---|---|
| | S. aureus | B. subtilis | aeruginosa | E. coli | E. faecalis | S. aureus | E. faecalis |
| | ATCC | ATCC | ATCC | ATCC | ATCC | B1 | W1 |
| | 25923 | 6633 | 27853 | 25922 | 29212 | (MRSA) | (VRE) |
| FIG. 1D | > 256 | >256 | >256 | >256 | >256 | >256 | >256 |
| FIG. 1E | > 256 | >256 | >256 | >256 | >256 | >256 | >256 |
| FIG. 1G | > 256 | >256 | >256 | >256 | >256 | >256 | >256 |
| FIG. 1H | > 256 | >256 | >256 | >256 | >256 | >256 | >256 |
| (eq. i) | | | | | | | |
| FIG. 1H | > 256 | >256 | >256 | >256 | >256 | >256 | >256 |
| (eq. ii) | | | | | | | |
| FIG. 1H | >256 | >256 | >256 | >256 | >256 | >256 | >256 |
| (eq. iii) | | | | | | | |
| FIG. 1H | >256 | >256 | >256 | >256 | >256 | >256 | >256 |
| (eq. iv) | | | | | | | |
| FIG. 1I | >256 | >256 | >256 | >256 | >256 | >256 | >256 |
| (eq. v) | | | | | | | |
| FIG. 1I | >256 | >256 | >256 | >256 | >256 | >256 | >256 |
| (eq. vi) | | | | | | | |
| FIG. 1I | >256 | >256 | >256 | >256 | >256 | >256 | >256 |
| (eq. vii) | | | | | | | |
| FIG. 1I | >256 | >256 | >256 | >256 | >256 | >256 | >256 |
| (eq. viii) | | | | | | | |
| FIG. 1I | 8 | 128 | >256 | >256 | 8 | 8 | 8 |
| (eq. ix) | | | | | | | |
| FIG. 1I | >256 | >256 | >256 | >256 | >256 | >256 | >256 |
| (eq. x) | | | | | | | |

TABLE 3
Minimum bactericidal concentration-MBC-values (μg/ml) against several bacterial
reference and multidrug-resistant strains.

| | | | P. | | | | |
|---|---|---|---|---|---|---|---|
| | S. aureus | B. subtilis | aeruginosa | E. coli | E. faecalis | S. aureus | E. faecalis |
| | ATCC | ATCC | ATCC | ATCC | ATCC | B1 | W1 |
| | 25923 | 6633 | 27853 | 25922 | 29212 | (MRSA) | (VRE) |
| FIG. 1D | — | — | — | — | — | — | — |
| FIG. 1E | — | — | — | — | — | — | — |
| FIG. 1G | — | — | — | — | — | — | — |
| FIG. 1H | — | — | — | — | — | — | — |
| (eq. i) | | | | | | | |
| FIG. 1H | — | — | — | — | — | — | — |
| (eq. ii) | | | | | | | |
| FIG. 1H | — | — | — | — | — | — | — |
| (eq. iii) | | | | | | | |
| FIG. 1H | — | — | — | — | — | — | — |
| (eq. iv) | | | | | | | |
| FIG. 1I | — | — | — | — | — | — | — |
| (eq. v) | | | | | | | |
| FIG. 1I | — | — | — | — | — | — | — |
| (eq. vi) | | | | | | | |
| FIG. 1I | — | — | — | — | — | — | — |
| (eq. vii) | | | | | | | |
| FIG. 1I | — | — | — | — | — | — | — |
| (eq. viii) | | | | | | | |
| FIG. 1I | 16 | >256 | — | — | 32 | 32 | 32 |
| (eq. ix) | | | | | | | |
| FIG. 1I | — | — | — | — | — | — | — |
| (eq. x) | | | | | | | |

Examination of the structures of fiscalin A (compound of FIG. 1I, equation vii), epi-fiscalin A (compound of FIG. 1I, equation viii), neofiscalin A (compound of FIG. 1I, equation ix) and epi-neofiscalin A (compound of FIG. 1I, equation x) revealed that they are diastereoisomers, differing from each other only in the absolute configuration of C-3 and C-22. However, the structures of the epimeric fiscalin C (compound of FIG. 1I, equation v) and epi-fiscalin C (compound of FIG. 1I, equation vi) have two methyl groups instead of one methyl group on C-22 as in the structures of the fiscalin A series. The fact that only neofiscalin A was active among the fiscalin analogs led to the conclusion that not only the substituent on C-22 (one methyl group) but also the stereochemistry of C-3 and C-22 (3R, 22R) may be required for the antibacterial activity.

From the combination of compounds of FIGS. 1D, 1E, 1G, 1H (equation i-iv), and 1I (equations v-x) with antibiotics, towards which the tested bacteria were resistant, fiscalin C (FIG. 1I, equation v) exhibited potential synergy with both ampicillin and oxacillin against the MRSA isolate (S. aureus B1). When not in combination, neither fiscalin C, ampicillin or oxacillin produced any zone of inhibition on agar plates. However, when fiscalin C was used in combination with ampicillin and oxacillin an inhibition zone was observed, of 8 and 10 mm, respectively. Therefore, there was an apparent synergistic effect, especially between fiscalin C and oxacillin.

To confirm these potential synergies, a checkerboard assay was performed by combining each antibiotic (ampicillin or oxacillin) with fiscalin C. From FIC index values shown in Table 4, it can be observed that the association of fiscalin C with oxacillin showed a clear synergistic effect (FIC index<0.5), while the combination with ampicillin was indifferent. These results may indicate that fiscalin C and oxacillin may somehow interact. This interaction needs to be further studied to identify how the synergy occurs. Noteworthy, the structural features required for such synergy may be different from those needed for antibacterial activity. As only fiscalin C exhibited a synergistic effect, it might be concluded that the presence of two methyl groups on C-22 and the 3S configuration are required for this activity.

TABLE 4
MIC values of fiscalin C in combination with
oxacillin and ampicillin and respective FIC indexes
obtained from the Checkerboard method.

| | MIC (μg/ml) | | | | |
|---|---|---|---|---|---|
| | | | Fiscalin C + | Oxacillin + | FIC |
| Strain | Fiscalin C | Oxacillin | Oxacillin | Fiscalin C | Index |
| S. aureus | >512 | 128 | 16 | 16 | <0.156 |
| B1 | | | | | |
| | | | Fiscalin C + | AMP + | |
| | Fiscalin C | Ampicillin | Ampicillin | Fiscalin C | |
| S. aureus | >512 | 128 | 512 | 128 | >1 |
| B1 | | | | | |

If the FIC index is lower than 0.5, it is consider to have a synergistic effect.

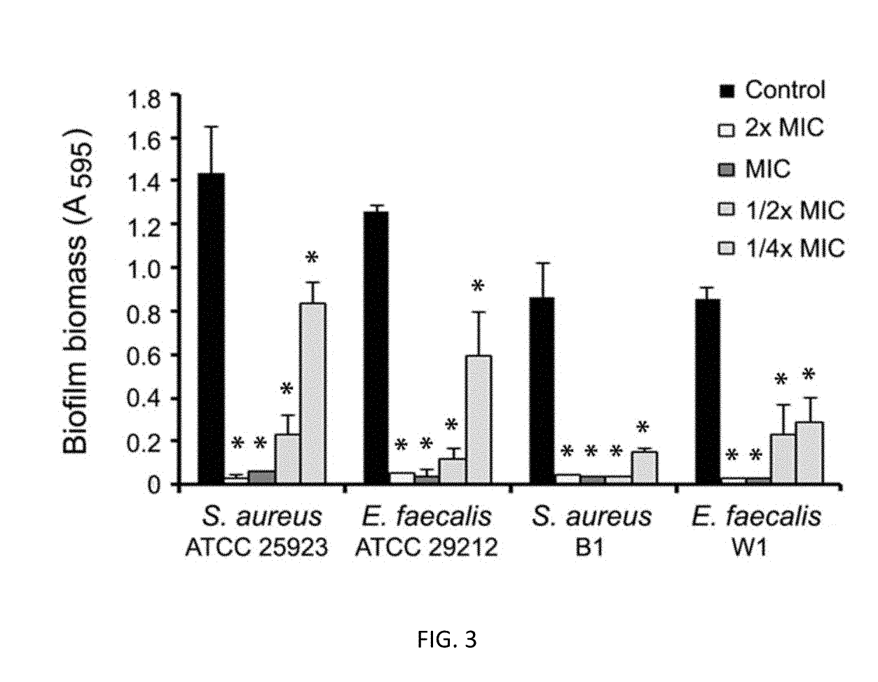

The effect of neofiscalin A on the biofilm formation of S. aureus ATCC 25923, E. faecalis ATCC 29212, S. aureus B1 and E. faecalis W1 was assessed at different concentrations (ranging from 2×MIC to ¼×MIC), through the biomass quantification after crystal violet staining and the results are shown in FIG. 3.

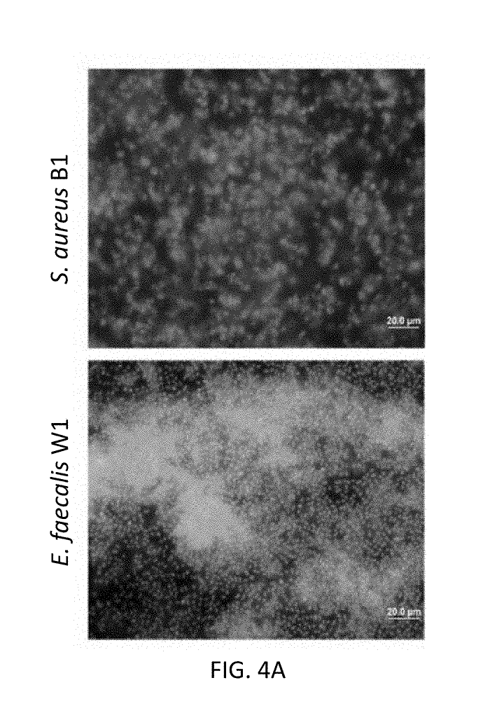

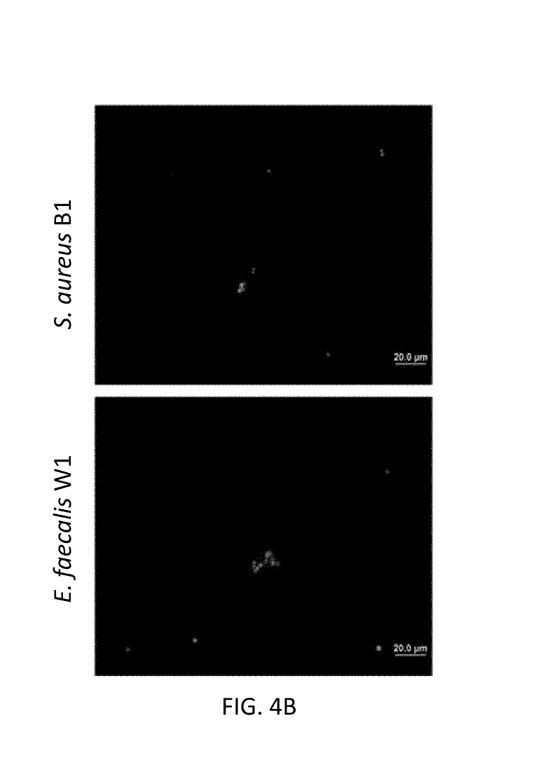

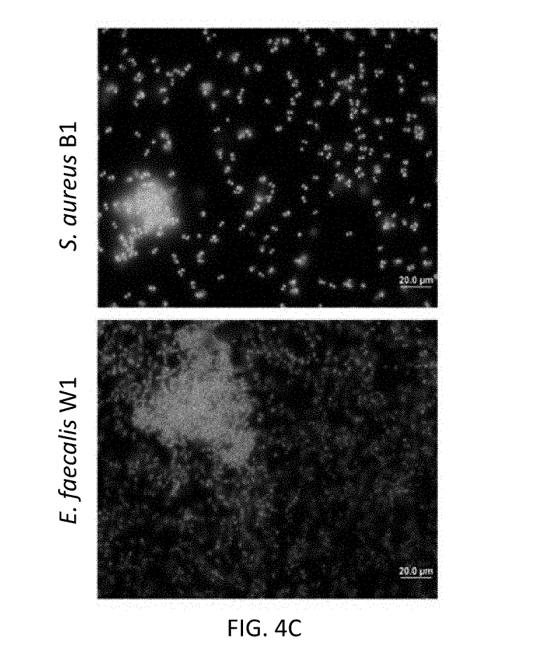

At 2×MIC and MIC of neofiscalin A, none of the strains was able to form a biofilm. At the sub-inhibitory concentration of ½×MIC, a small increase in the biomass was verified; however the strains did not succeed in forming much biofilm. A lower sub-inhibitory concentration of ¼×MIC allowed the formation of a larger biofilm, but smaller than that observed in the control group. In order to substantiate the effect of this compound on the biofilm formation, a microscopic visualization of the biofilm produced by S. aureus B1 and E. faecalis W1 was carried out using the Live/Dead staining, as shown in FIGS. 4A-4C. In the presence of neofiscalin A, at its MIC values, no growth was observed and consequently, no biofilm was produced (FIG. 4B). However, at the concentration of ¼×MIC a biofilm was formed (FIG. 4C), though it was not as large as in the control (FIG. 4A). In contrast, the sub-inhibitory concentrations of antibiotics, to which bacteria were continuously exposed, have been reported to greatly increase the biofilm formation, thus the neofiscalin A is an unexpected and efficient alternative to the antibiotics currently used.

The biofilm inhibitory concentration (BIC) value of neofiscalin A against 24-h biofilms of S. aureus B1 and E. faecalis W1 was 96 and 80 μg/ml, respectively. Nevertheless, the biofilm eradication concentration (BEC) values were found to be higher than 25×MIC, i.e., 200 μg/ml for both strains. However, higher concentrations could not be tested due to the limited quantity of this compound available to perform all these biological assays. Nevertheless, neofiscalin A showed great potential in interfering with the viability of preformed biofilms of S. aureus and E. faecalis. This result is surprising and unexpected since previous studies have shown that 24-h preformed staphylococcal biofilms subjected to treatment with 500 mg/I of tetracycline were not eradicated, once a significant part of the cells within the biofilms were still viable (Flemming, K., Klingenberg, C., Cavanagh, J. P., Sletteng, M., Stensen, W., Svendsen, J. S., et al. 2009. High in vitro antimicrobial activity of synthetic antimicrobial peptidomimetics against staphylococcal biofilms. J. Antimicrob. Chemother. 63:136-45).

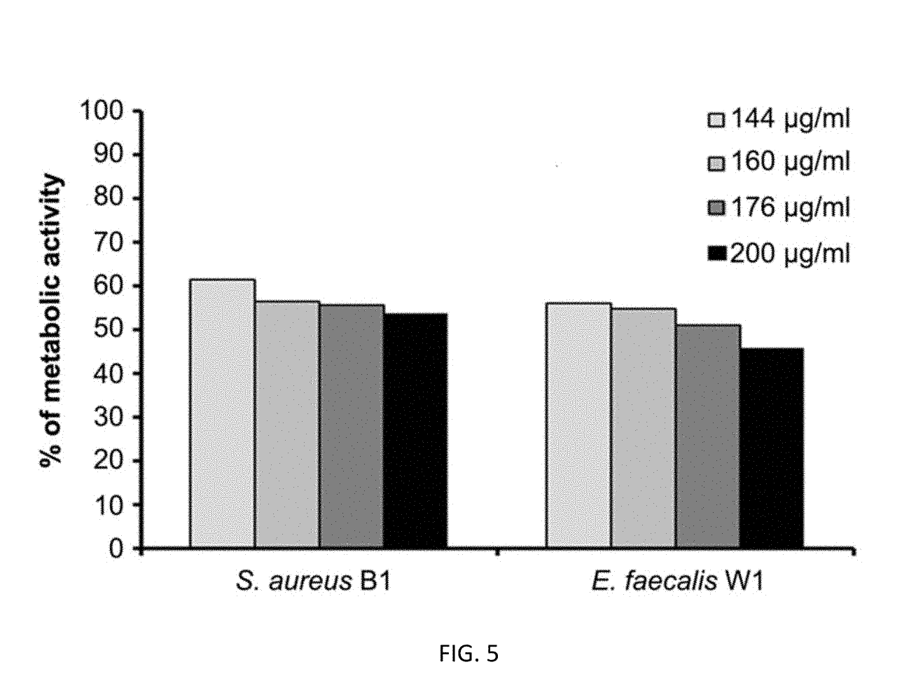

The results of the metabolic activity of biofilms, assessed by the MTT assay, after treatment with increasing concentrations of neofiscalin A are shown in FIG. 5.

The metabolic activity of 24-h biofilms of S. aureus B1 and E. faecalis W1 treated for 24 h with 200 μg/ml of neofiscalin A was reduced by nearly 50%.

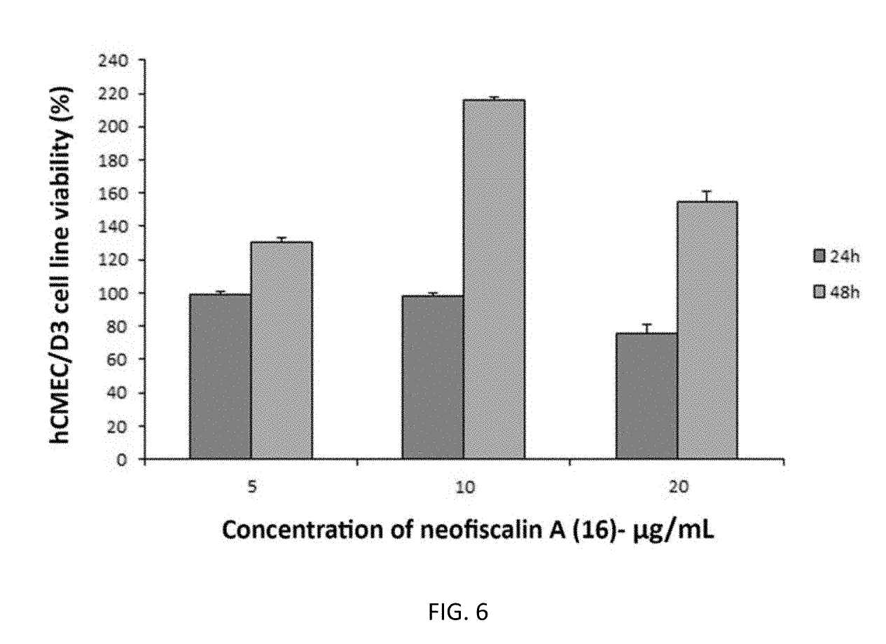

The cytotoxicity assay to assess the eventual toxicity of neofiscalin A, in particular, at concentrations of 20, 10 and 5 μg/ml to a human brain endothelial cell line (hCMEC/D3) revealed absence of cytotoxicity, i.e., the percentage of cell viability was equal or higher than the viability obtained in the negative control, DMSO (FIG. 6).

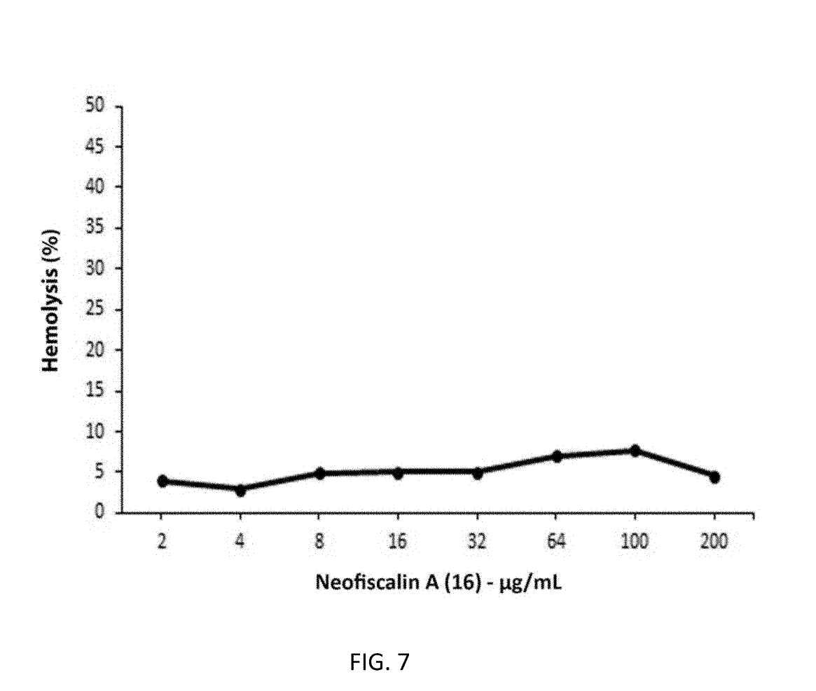

The hemolytic assay showed that neofiscalin A does not cause human blood hemolysis at its MIC value nor at higher values, such as 200 μg/ml. In particular, for the range of concentrations tested, the hemolytic activity was never higher than 8%. The dose-response curve for the hemolytic activity of neofiscalin A is shown in FIG. 7.

To sum up, neofiscalin A showed antibacterial activity against Gram-positive bacteria, including MRSA and VRE strains, with a MIC value of 8 μg/ml. Neofiscalin A was also found to inhibit the biofilm formation by those strains. The BIC values of neofiscalin A against the MRSA and VRE isolates was found to be 96 and 80 μg/ml, respectively. Moreover, neofiscalin A at a concentration of 200 μg/ml was able to reduce the metabolic activity of the biofilm by nearly 50%. Regarding the toxicity, neofiscalin A showed no cytotoxicity against a human cell line and no hemolytic activity against human red blood cells. Additionally, fiscalin C was shown to have a synergistic effect with oxacillin against the Gram-positive bacteria, in particular a MRSA isolate.

All references recited in this document are incorporated herein in their entirety by reference, as if each and every reference had been incorporated by reference individually.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in different embodiments of the invention, to the tenth of the unit of the lower limit of the range, unless the context clearly dictates otherwise. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

In addition, it is to be understood that any particular embodiment of the present invention may be explicitly excluded from any one or more of the claims. Where ranges are given, any value within the range may explicitly be excluded from any one or more of the claims. Any embodiment, element, feature, application, or aspect of the compositions and/or methods of the invention, can be excluded from any one or more claims.

The disclosure should not be seen in any way restricted to the embodiments described and a person with ordinary skill in the art will foresee many possibilities to modifications thereof.

The above described embodiments are combinable. The following claims further set out particular embodiments of the disclosure.

1. A compound of the following formula 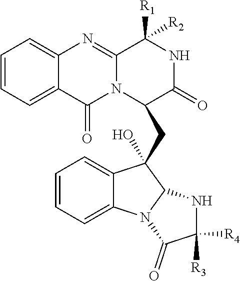
or a pharmaceutically acceptable salt, ester, solvate or prodrug thereof,
wherein
R1, R2, R3 and R4 are independently selected from the group consisting of: H, C1-C10 alkyl, C2-C10 alkenyl, and C1-C10 aryl; and
wherein R1 is equal to R3 or R3 is equal to R4, and
R2 is selected from the group consisting of C1-C10 alkyl.

2. The compound of claim 1, wherein the compound exhibits antibacterial activity against Gram-positive bacterium.

3. The compound of claim 2, wherein the Gram-positive bacterium is selected from the group consisting of: Staphylococcus spp., Streptococcus spp., Enterococcus spp., Listeria spp., Clostridium spp., Corynebacterium spp., Nocardia spp., Bacillus spp., and mixtures thereof.

4. The compound of claim 1, wherein the alkyl is C1-C6.

5. The compound of claim 1, wherein the alkyl is a non-linear alkyl.

6. The compound of claim 1, wherein R1 and R3 are H.

7. The compound of claim 1, wherein R3 and R4 are CH3.

8. The compound of claim 1, wherein R1 and R3 are H, R2 is CH(CH3)2 and R4 is CH3.

9. The compound of claim 1, wherein R1 is H, R2 is CH(CH3)2, R4 and R3 are CH3.

10. A composition comprising:
the compound of claim 1 in a therapeutically effective amount; and
a pharmaceutically acceptable excipient.

11. The composition of claim 10, further comprising an antibiotic.

12. The composition of claim 11, wherein the antibiotic is a beta-lactam.

13. The composition of claim 11, wherein for the compound of claim 1, R1 is H, R2 is CH(CH3)2, and R4 and R3 are CH3.

14. The composition of claim 12, wherein the beta-lactam antibiotic is selected from the group consisting of: oxacillin, nafcillin, cloxacillin, dicloxacillin, flucloxacillin, and mixtures thereof.

15. The composition of claim 11, wherein the composition is suitable for topical, oral, parental, or injectable administration.

16. A method for the treatment of a patient having a bacterial infection, the method comprising:
administering an effective amount of the composition of claim 10 to the patient.

17. The method of claim 16, wherein the composition is administered with a beta-lactam antibiotic.

18. (canceled)

19. The compound of claim 1, wherein the alkyl is C1-C3.
